(19) United States
(12) Patent Application Publication
(10) Pub. No.: US 2021/0030979 A1
(43) Pub. Date: Feb. 4, 2021

(54) SYSTEMS AND METHODS FOR CONTROLLING THE OPERATION OF PULMONARY DRUG DELIVERY DEVICES AND OTHER INHALATION DEVICES

(71) Applicant: EField Innovations, LLC, Columbus, OH (US)

(72) Inventors: William C. Zimlich, JR., North Royalton, OH (US); Brian Lipp, Columbus, OH (US); King W. Wong, Columbus, OH (US)

(21) Appl. No.: 16/942,915

(22) Filed: Jul. 30, 2020

Related U.S. Application Data
(60) Provisional application No. 62/880,929, filed on Jul. 31, 2019.

(51) Int. Cl. A61M 15/02; G16H 20/13; G16H 40/67

(52) U.S. Cl. CPC A61M 15/02; G16H 20/13; G16H 40/67; A61M 2205/502; A61M 2205/52; A61M 2205/8206; A61M 2205/587; A61M 2205/3584

(57) ABSTRACT

A system for operating an inhalation device including an inhalation device that has been configured to store at least one substance and dispense the substance in a predetermined manner, wherein the inhalation device is configured to gather usage information from the inhalation device and transmit the gathered usage information to a location remote from the inhalation device and to receive operational commands from a location remote from the inhalation device; a controller in communication with the inhalation device, wherein the controller is configured to receive usage information from the inhalation device and to transmit operational commands to the inhalation device; and a remote server in communication with the controller, wherein the remote server is configured to receive queries from the controller, and transmit information relevant to the queries back to the controller.

### CROSS-REFERENCE TO RELATED APPLICATIONS

This patent application claims the benefit of U.S. Provisional Patent Application Ser. No. 62/880,929 filed on Jul. 31, 2019 and entitled “Systems and Methods for Controlling the Operation of Pulmonary Drug Delivery Devices and Other Inhalation Devices”, the disclosure of which is hereby incorporated by reference herein in its entirety and made part of the present U.S. utility patent application for all purposes.

### BACKGROUND

Disclosed implementations relate in general to drug delivery systems, devices and methods, and more specifically to a system and method for remotely controlling or affecting the operation or functionality of pulmonary drug delivery devices and other types of inhalers utilizing electrohydrodynamic (EHD) atomization processes or other aerosol-based processes.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

In one implementation, a first system for controlling the operation of an inhalation device is provided. This system comprises an inhalation device, wherein the inhalation device has been configured to store at least one substance and dispense the at least one substance in a predetermined manner; and wherein the inhalation device is configured to gather usage information from the inhalation device and transmit the gathered usage information to a location or device remote from the inhalation device and to receive operational commands from a location or device remote from the inhalation device; a controller in electrical or digital communication with the inhalation device, wherein the controller is configured to receive usage information from the inhalation device and to transmit operational commands to the inhalation device; and a remote server in electrical or digital communication with the controller, wherein the remote server is configured to receive queries from the controller, and transmit information relevant to the queries back to the controller. The inhalation device may be an aerosol inhalation device. The inhalation device may be an electrohydrodynamic aerosolization device. The at least one substance may be a drug. The at least one substance may be a cannabis-based product. The operational commands may include dosage information. The controller may be smartphone or tablet and the remote server may be in electrical or digital communication with one or more product related networks, medical networks, or health care provider networks.

In another implementation, a second system for controlling the operation of an inhalation device is provided. This system comprises an aerosol inhalation device, wherein the aerosol inhalation device has been configured to store at least one substance and dispense the at least one substance in a predetermined manner; and wherein the aerosol inhalation device is configured to gather usage information from the aerosol inhalation device and transmit the gathered usage information to a location or device remote from the aerosol inhalation device and to receive operational commands from a location or device remote from the aerosol inhalation device; a controller in electrical or digital communication with the aerosol inhalation device, wherein the controller is configured to receive usage information from the aerosol inhalation device and to transmit operational commands to the aerosol inhalation device; and a remote server in electrical or digital communication with the controller, wherein the remote server is configured to receive queries from the controller, and transmit information relevant to the queries back to the controller. The inhalation device may be an electrohydrodynamic aerosolization device. The at least one substance may be a drug. The at least one substance may be a cannabis-based product. The operational commands may include dosage information. The controller may be a smartphone or tablet. The remote server may be in electrical or digital communication with one or more product related networks, medical networks, or health care provider networks.

In still another implementation, a third system for controlling the operation of an inhalation device is provided. This system comprises an electrohydrodynamic aerosolization device, wherein the electrohydrodynamic aerosolization device has been configured to store at least one substance and dispense the at least one substance in a predetermined manner; and wherein the electrohydrodynamic aerosolization device is configured to gather usage information from the electrohydrodynamic aerosolization device and transmit the gathered usage information to a location or device remote from the electrohydrodynamic aerosolization device and to receive operational commands from a location or device remote from the electrohydrodynamic aerosolization device; a controller in electrical or digital communication with the electrohydrodynamic aerosolization device, wherein the controller is configured to receive usage information from the electrohydrodynamic aerosolization device and to transmit operational commands to the electrohydrodynamic aerosolization device; and a remote server in electrical or digital communication with the controller, wherein the remote server is configured to receive queries from the controller, and transmit information relevant to the queries back to the controller. The at least one substance may be a drug. The at least one substance may be a cannabis-based product. The operational commands may include dosage information. The controller may be a smartphone or tablet, wherein the smartphone or tablet is in electrical or digital communication with one or more product related networks, medical networks, or health care provider networks.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed inventive subject matter and, together with the general description given above and detailed description given below, serve to explain the principles of the disclosed subject matter, and wherein:

FIG. 1 is a diagram depicting the basic components of an example system for controlling the operation of pulmonary drug delivery devices and other inhalation devices;

FIG. 2 is a diagram depicting the controller component and one of the reciprocal communication components of the example system of FIG. 1;

FIG. 3 is a diagram depicting the device component of the example system of FIG. 1, wherein the subsystems and subcomponents of the device component are shown;

FIG. 4 is a unified modeling language (UML) sequence diagram depicting an example sequence for initializing the device to receive a prescription; and

FIG. 5 is a unified modeling language (LAI) sequence diagram depicting locking the device and then unlocking the device such that aerosolization is controlled based on authentication of user identity.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed inventive subject matter. Accordingly, the following implementations are set forth without any loss of generality to, and without imposing limitations upon, the claimed subject matter.

As previously stated, disclosed example implementations relate in general to drug delivery systems, devices and methods, and more specifically to systems, devices, and methods for remotely controlling or affecting the operation or functionality of pulmonary drug delivery devices and other types of inhalers utilizing electrohydrodynamic (EHD) atomization processes or other aerosol-based processes. Disclosed aerosolization devices include pulmonary drug delivery devices and inhalers utilizing specific electrohydrodynamic (EHD) atomization processes such as those disclosed in U.S. Pat. Nos. 7,931,020 and 7,849,850, both of which form part of this disclosure and are included herein, in their entirety, in the appendix attached hereto. These systems and devices also include data collection capabilities as well as capabilities that include the communication of the collected data using various wireless technologies such as, for example, Bluetooth, Zigbee, IR, and the like.

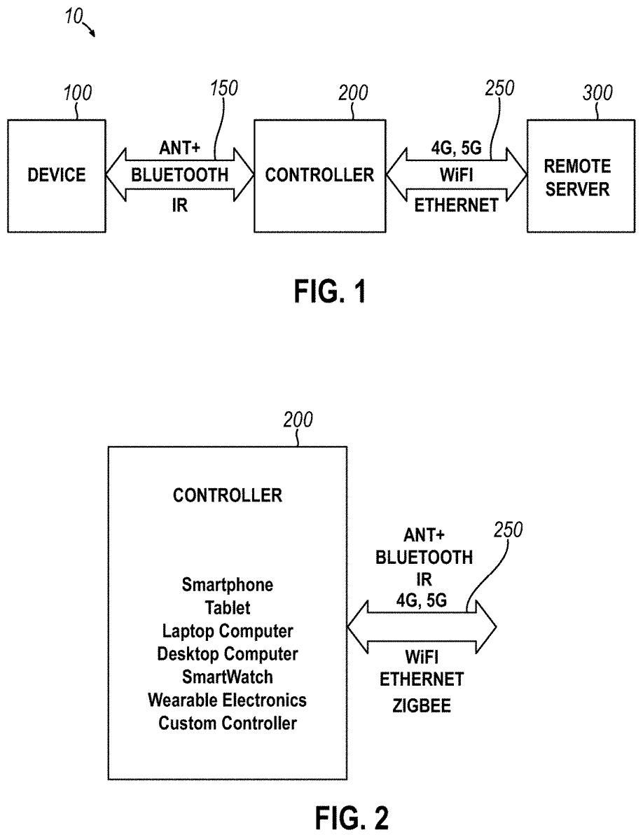

Example implementations of the disclosed system, device, and method include three basic components: (i) an inhalation device; (ii) a controller in two-way communication with the inhalation device; and (iii) a remote server in two-way communication with the controller. FIG. 1 depicts these basic system components as well as the reciprocal interfaces between these components. With reference to FIG. 1, drug delivery system 10 includes aerosolization device 100; controller 200; and remote server 300. In the example depicted in FIG. 1, reciprocal communication interface 150 between device 100 and controller 200 may include ANT+, Bluetooth, InfraRed (IR), or any other compatible communication protocol. Controller 200 is typically computer-based and remote server 300 typically utilizes an Internet service that enables reciprocal communication 250 with controller 200. Example communication protocols that may be used for reciprocal communication 250 include HTTP, HTTPS, MQTT or any other compatible and acceptable communication protocol. Remote server 300 may include one or more physical servers, virtual cloud-based servers, or any other suitable server type or configuration.

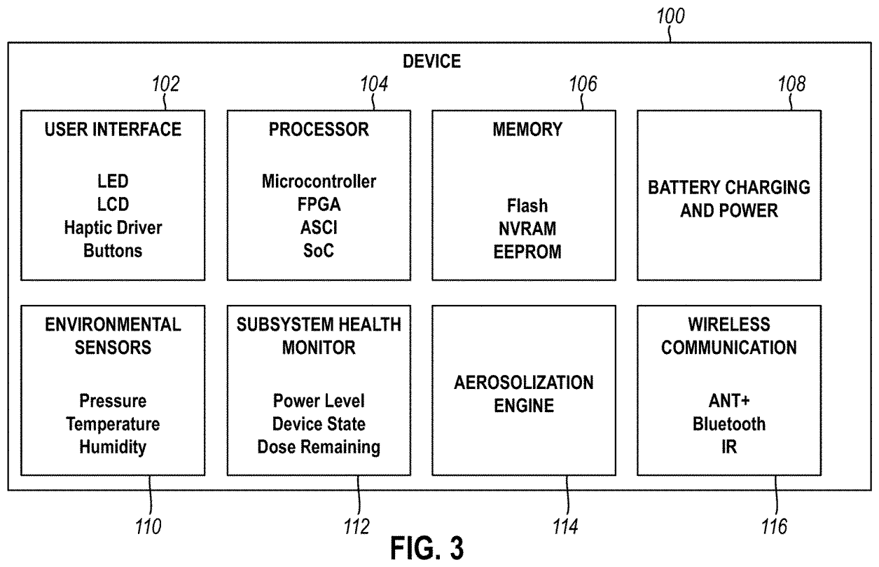

FIG. 2 provides a diagram depicting the controller component and one of the reciprocal communication components of the example system of FIG. 1. Non-limiting examples of devices that may be used for controller 200 include commercial off the shelf smartphones, tablet computers, laptop computers, desktop computers, smartwatches, and various wearable electronics. Controller 200 may also be a custom electronic device capable of communicating with remote server 300. By way of example, reciprocal communication 250 may include the use of ANT+; Bluetooth; InfraRed (IR); cellular networks such as 4G, 5G; wireless communications (WiFi); wireless local area network (WLAN); wired ethernet; and Zigbee.

FIG. 3 provides a diagram depicting the device component of the example system of FIG. 1, wherein the subsystems and subcomponents of device component 100 are shown. These subsystems and subcomponents may include user interface 102; processor 104; memory 106; battery charging and power 108; environmental sensors 110; subsystem health monitor 112; aerosolization engine 114; and wireless communications 116. User interface 102 may include light-emitting diodes (LED), liquid crystal displays (LCD), buttons, breath sensors, and/or haptic (vibration) drivers. Processor 104 may include microprocessors, microcontrollers, digital signal processors (DSP), field-programmable gate arrays (FPGA), application specific integrated circuits (ASIC), system on chip (SoC) or equivalent discrete or analog circuitry, or various combinations thereof. The functions attributed to processor 104 may be referred to as software, firmware, hardware, gateware or any combination thereof. Storage memory 106 may include any volatile, non-volatile media such as flash memory, random access memory (RAM), read-only memory (ROM), non-volatile RAM (NVRAM), electrically erasable programmable ROM (EEPROM) or any other media. Storage memory 106 may be configured to store operational parameters, data from subsystems, usage information, prescription parameters, dosage information, and various other information related to therapy and treatment. Battery charging and power 108 includes power conversion and battery charging circuitry.

Environmental sensors 110 may include pressure, temperature and humidity sensors. Subsystem health monitor 112 monitors the various subsystems of the device including, for example, power levels, sub-system states, and dose remaining. Aerosolization engine 114 delivers desired aerosolization dosages. Communication unit 116 may include any suitable hardware, firmware, gateware, software, or any combination thereof for communicating with controller 200. Communication unit 116 may be configured to transfer data using standard protocols such as ANT+, Bluetooth, IR, or any other compatible protocol. Reciprocal communication interface 150 may be configured to switch between two or more protocols that have different data transmission rates and different power consumption rates. For example, if Bluetooth is used, communication may switch between Bluetooth base rate/enhanced data rate (BR/EDR) and Bluetooth Low Energy (BLE) protocols depending on volume of data to be transmitted and on power capability. Controller 200 may both transmit and receive data and/or commands to and/or from device 100. Either controller 200 or device 100 may periodically initiate communications with the other to see if any data should be transferred or if various commands are to be processed. Controller 200 may periodically communicate with remote server 300 to determine whether commands should be relayed to or from device 100.

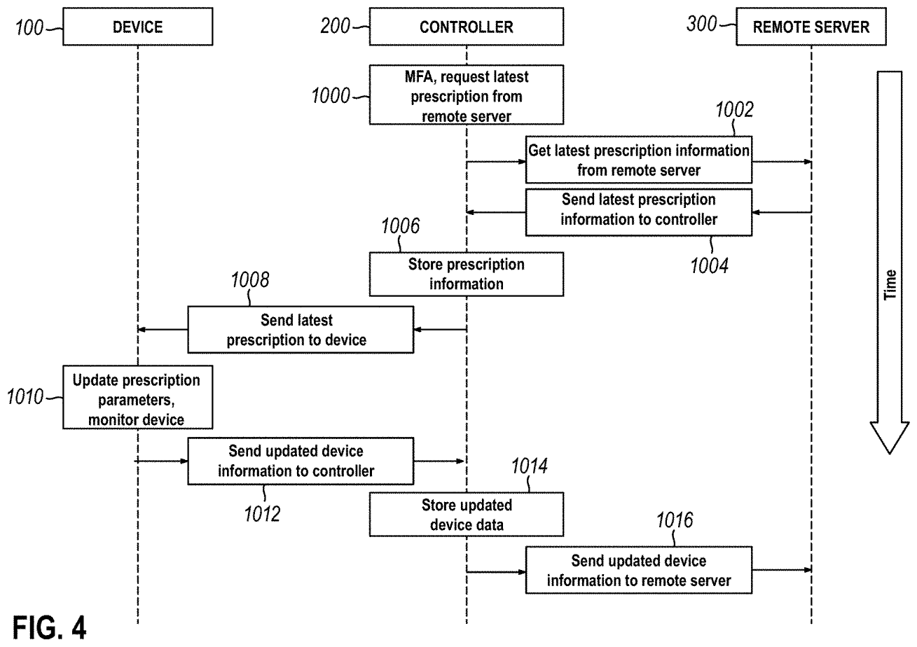

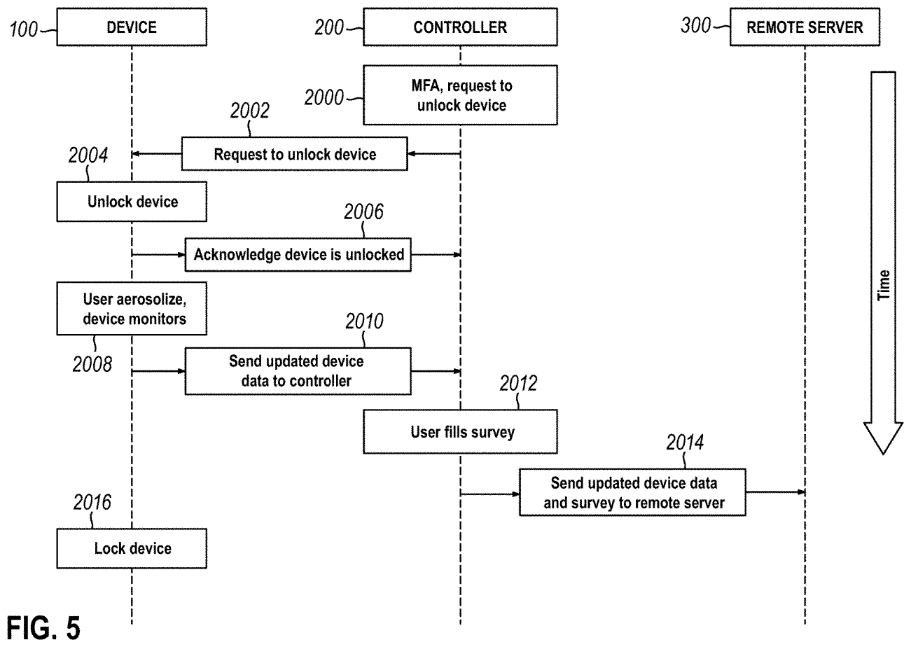

FIGS. 4 and 5 illustrate example interactions between aerosolization device 100, controller 200, and remote server 300 using unified modeling language (UML) sequence diagrams. The downward vertical aspect of each diagram represents time. The horizontal aspect of each diagram illustrates the exchange of information between aerosolization device 100, controller 200, and remote server 300. These diagrams describe information exchange at a high level and do not depict intermediate layers such as encryption or a protocol specific layer, or lower layers that are communication specific (e.g. Bluetooth). These diagrams also do not show all possible hand-shaking protocols. In these examples, controller 200 is a smartphone having biometric sensors.

FIG. 4 provides a unified modeling language (UML) sequence diagram depicting an example sequence for initializing device 100 to receive a prescription. In this example, the sequence starts with a request (1000) by a user of the system to receive their most recent prescription. The request may be made through dedicated application software located on the smartphone. The user is first required to use multi-factor authentication (MFA) to verify their identity. This may include the use of a fingerprint, face scan, or password. Once MFA is verified, controller 200 communicates with remote server 300 for receiving the most recent prescription information for device 100 (1002). Remote server 300 then retrieves the relevant prescription information for device 100 and communicates this information to controller 200 (1004). The prescription information may include, for example, dosage level per aerosolization, maximum number or aerosolization events in a predetermined time period, and/or minimum time between aerosolization events. Controller 200 stores the prescription information received from remote server 300 (1006) and then transmits the prescription information to device 100 (1008), which receives the information and stores the parameters. Device 100 then monitors the device subsystems and saves subsystem status data (1010). Device 100 then communicates the updated device information to controller 200 (1012), which stores the updated device information (1014). Updated device information is then transmitted to remote server 300 (1016).

For some active ingredients (e.g., narcotics, opioids, and cannabis-based products, including CBD, CBG, and THC), device 100 can be locked so that it cannot be aerosolized without further authentication of user identity. FIG. 5 provides a unified modeling language (UML) sequence diagram depicting locking device 100 and then unlocking device 100 such that aerosolization is controlled based on authentication of user identity. In this example, controller 200 is a smartphone having biometric sensors. A request is made by a user to unlock device 100 for a predetermined period of time (2000). The request may be made through dedicated application software located on the smartphone. The user is first required to use multi-factor authentication (MFA) to verify their identity. This may include the use of a fingerprint, face scan, or password. Once MFA is verified, prescription parameters are checked to determine if the user is authorized to dose. If the dose level and time window are consistent with permitting the user to aerosolize, controller 200 instructs device 100 to unlock and device 100 unlocks for a predetermined period of time and/or dosage limit (2004). Device 100 then communicates to controller 200 that device 100 has been successfully unlocked (2006) and device 100 may be aerosolized by the user (2008). After aerosolization, device 100 transmits updated device data to controller 200 (2010). Controller 200 then provides the user with a survey (2012) for submitting information regarding drug effects, effectiveness, and current health status. Once the survey has been completed, the updated device data and survey data are transmitted to remote server 300 (1014). After a predetermined period of time has passed, or a maximum permitted dose has been dispensed from device 100, device 100 is locked to prevent further use (2016).

Other examples of collected and transmitted data include patient usage history; compliance with prescribed or recommended dosages or amounts; quantity or frequency of doses; volume or amount of formulation or active ingredient delivered to patient by device; formulation lot information; and any other data or information that is useful to a patient, physician, or caregiver. The disclosed pulmonary drug delivery devices and inhalers are or may be in electrical or digital communication with a device such as a smartphone that includes a visual display and interface. Through this type of interface, queries may be directed toward a user of the device including questions regarding drug effects and effectiveness and questions regarding current health status of the user. System and device capabilities include tracking usage, thereby facilitating automatic on-line ordering of refill drug cartridges. Because EHD is primarily an electrical process and not a mechanical aerosolization method as in other pulmonary delivery methods, any desired sensors and controls may be implemented and included with relative ease.

Various implementations of the disclosed systems and devices provide the combination of a highly repeatable, precise, rapid onset delivery method such as EHD with data collection and wireless communication to a mobile device or network. This combination facilitates improvements to the current standard of care for inhaled drug delivery for both patients and consumers by providing: (i) more precise, real time feedback and traceability regarding device usage; and (ii) the source and amount of active ingredient delivered to the patient or consumer. The collection of such data combined with user responses to validated questions provides valuable insight regarding next generation drug/device development.

Among other advantages, uses, and functions, patient and user compliance with prescriptions or recommended dose limits can be discretely managed with the disclosed systems and devices. Other available inhalers or inhalation device technologies exhibit slower or more gradual pharmacokinetic profiles, thereby resulting in a longer delay between when a substance or composition is delivered and when relief or the desired result is obtained by the user. Accordingly, with other inhalation devices, a patient or user may dose themselves multiple times to get the same relief or derive the same benefit, thus possibly resulting in overdosing. The combination of precise repeatable delivery with a method of communicating to the user that the appropriate or maximum dosage has been reached is a specific advantage provided by the disclosed systems and devices. Additionally, this information may be communicated to caregivers, doctors, and other parties as an aspect of managing the patient care.

The disclosed systems and methods are particularly useful for applications wherein real-time feedback of dose frequency and dosage amounts are important, and wherein traceability of active ingredients (e.g., as narcotics, opioids, and cannabis-based products, including CBD, CBG, and THC) is of concern. As the risk of counterfeit drugs and supplements increases, the traceability of an active ingredient or formulation provided by the disclosed systems and methods may be used to prevent or greatly reduce the introduction, distribution, and use of counterfeit drugs or supplements.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about” used throughout this specification are used to describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

It should be appreciated that all combinations of the foregoing concepts and additional concepts discussed in greater detail herein (provided such concepts are not mutually inconsistent) are contemplated as being part of the disclosed inventive subject matter. In particular, all combinations of claimed subject matter appearing at the end of this disclosure are contemplated as being part of the inventive subject matter disclosed herein. While the disclosed inventive subject matter has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed inventive subject matter in its broader aspects is not limited to any of the specific details, representative devices and methods, and/or illustrative examples shown and described. Accordingly, departures may be made from such details without departing from the spirit or scope of the general inventive concept.

What is claimed:

1. A system for controlling the operation of an inhalation device, comprising:
(a) an inhalation device,
(i) wherein the inhalation device has been configured to store at least one substance and dispense the at least one substance in a predetermined manner; and
(ii) wherein the inhalation device is configured to
a) gather usage information from the inhalation device and transmit the gathered usage information to a location or device remote from the inhalation device, and
b) receive operational commands from a location or device remote from the inhalation device; and
(b) a controller in electrical or digital communication with the inhalation device, wherein the controller is configured to
(i) receive usage information from the inhalation device, and
(ii) transmit operational commands to the inhalation device; and
(c) a remote server in electrical or digital communication with the controller, wherein the remote server is configured to
(i) receive queries from the controller, and
(ii) transmit information relevant to the queries back to the controller.

2. The system of claim 1, wherein the inhalation device is an aerosol inhalation device.

3. The system of claim 1, wherein the inhalation device is an electrohydrodynamic aerosolization device.

4. The system of claim 1, wherein the at least one substance is a drug.

5. The system of claim 1, wherein the at least one substance is a cannabis-based product.

6. The system of claim 1, wherein the operational commands include dosage information.

7. The system of claim 1, wherein the controller is a smartphone or tablet.

8. The system of claim 1, wherein the remote server is in electrical or digital communication with one or more product related networks, medical networks, or health care provider networks.

9. A system for controlling the operation of an inhalation device, comprising:
(a) an aerosol inhalation device,
(i) wherein the aerosol inhalation device has been configured to store at least one substance and dispense the at least one substance in a predetermined manner; and
(ii) wherein the aerosol inhalation device is configured to
a) gather usage information from the aerosol inhalation device and transmit the gathered usage information to a location or device remote from the aerosol inhalation device, and
b) receive operational commands from a location or device remote from the aerosol inhalation device; and
(b) a controller in electrical or digital communication with the aerosol inhalation device, wherein the controller is configured to
(i) receive usage information from the aerosol inhalation device, and
(ii) transmit operational commands to the aerosol inhalation device; and
(c) a remote server in electrical or digital communication with the controller, wherein the remote server is configured to
(i) receive queries from the controller, and
(ii) transmit information relevant to the queries back to the controller.

10. The system of claim 9, wherein the inhalation device is an electrohydrodynamic aerosolization device.

11. The system of claim 9, wherein the at least one substance is a drug.

12. The system of claim 9, wherein the at least one substance is a cannabis-based product.

13. The system of claim 9, wherein the operational commands include dosage information.

14. The system of claim 9, wherein the controller is a smartphone or tablet.

15. The system of claim 9, wherein the remote server is in electrical or digital communication with one or more product related networks, medical networks, or health care provider networks.

16. A system for controlling the operation of an inhalation device, comprising:
(a) an electrohydrodynamic aerosolization device,
(i) wherein the electrohydrodynamic aerosolization device has been configured to store at least one substance and dispense the at least one substance in a predetermined manner; and
(ii) wherein the electrohydrodynamic aerosolization device is configured to
a) gather usage information from the electrohydrodynamic aerosolization device and transmit the gathered usage information to a location or device remote from the electrohydrodynamic aerosolization device, and
b) receive operational commands from a location or device remote from the electrohydrodynamic aerosolization device; and
(b) a controller in electrical or digital communication with the electrohydrodynamic aerosolization device, wherein the controller is configured to
(i) receive usage information from the electrohydrodynamic aerosolization device, and
(ii) transmit operational commands to the electrohydrodynamic aerosolization device; and
(c) a remote server in electrical or digital communication with the controller, wherein the remote server is configured to
(i) receive queries from the controller, and
(ii) transmit information relevant to the queries back to the controller.

17. The system of claim 16, wherein the at least one substance is a drug.

18. The system of claim 16, wherein the at least one substance is a cannabis-based product.

19. The system of claim 16, wherein the operational commands include dosage information.

20. The system of claim 16, wherein the controller is a smartphone or tablet, and wherein the smartphone or tablet is in electrical or digital communication with one or more product related networks, medical networks, or health care provider networks.
